(19) United States
(12) Patent Application Publication
(10) Pub. No.: US 2016/0256172 A1
(43) Pub. Date: Sep. 8, 2016

(54) VERTEBRAL ENDPLATE APPARATUS AND METHOD

(71) Applicant: Scott L. Blumenthal, Frisco, TX (US)

(72) Inventors: Scott L. Blumenthal, Frisco, TX (US); Lisa Ferrara, Southport, NC (US)

(21) Appl. No.: 14/910,145

(22) PCT Filed: Aug. 4, 2014
(86) PCT No.: PCT/US14/49595

§ 371 Date: Feb. 4, 2016

Related U.S. Application Data
(60) Provisional application No. 61/862,149, filed on Aug. 5, 2013.

(51) Int. Cl. A61B 17/16

(52) U.S. Cl. CPC A61B 17/1631; A61B 17/1604; A61B 17/1671; A61B 2217/007

(57) ABSTRACT

A surgical apparatus for use in connection with spinal surgery. The apparatus has a first portion having a tip for first following along an insertion axis, for entering into a spinal disc, and for penetrating an endplate of a vertebra adjacent the spinal disc. The apparatus also has a second portion, adjacent the first portion, for remaining along the insertion axis as the tip of the first portion extends to penetrate the endplate of the vertebra adjacent the disc.

### TECHNICAL FIELD

The preferred embodiments relate to relate to spinal surgical apparatus and methodology and more particularly to accessing spinal biological materials via a vertebral endplate.

### BACKGROUND ART

Spinal pain and more particularly pain in connection with spinal discs may occur from both acute and chronic conditions as well as with deterioration from disease and age. Such disc issues arise most commonly in the spinal lumbar region, second most commonly in the cervical region, and finally least commonly in the thoracic region. Treatment for such issues may include medication (e.g., anti-inflammatories, steroids, cortisone, and pain therapies) as well as surgery. Contemporary surgeries are typically directed at reducing pain in association with a damaged or diseased disc, such as by reducing pressure in the area, removing damaged portions of the disc, replacing the disk with an artificial counterpart, and/or bracing the spine in the area of the suspected disc through the use of spinal fusion, that is, fusing together the vertebrae that are located above and below the disc(s) at issue. Such approaches have some measure of success in some patients, but also carry considerable cost, risk of complications, and in some instances limited or no pain relief.

By way of further context, FIG. 1 illustrates a simplified view of two spinal vertebrae, where the spine is generally comprised of numerous vertebrae that span downward from the neck (or cervical) region, through the thoracic region and the lumbar region, to the sacrum. The preferred embodiments may have preferred application to surgery in the lumbar region, but such embodiments, or alternative preferred embodiments, may prove beneficial for other regions of the spine (i.e., cervical; thoracic). The additional detail of FIG. 1 illustrates that each pair of vertebrae are separated by an intervertebral disc. More particularly, each vertebra has an upper and lower surface referred to as an endplate, and the disc is therefore located between the upper endplate of one vertebra and the lower endplate of a neighboring vertebra.

Given the preceding, the preferred embodiments relate to repairing injury, damage, or deterioration to a spinal disc and in relation to the endplate either above it or below it (or both), as further described below. Numerous benefits will be appreciated by one skilled in the art, given an understanding of that discussion and with additional observations to follow.

### DISCLOSURE OF INVENTION

In one preferred embodiment, there is a surgical apparatus for use in connection with spinal surgery. The apparatus comprises a first portion having a tip for first following along an insertion axis, for entering into a spinal disc, and for penetrating an endplate of a vertebra adjacent the spinal disc. The apparatus further comprises a second portion, adjacent the first portion, for remaining along the insertion axis as the tip of the first portion extends to penetrate the endplate of the vertebra adjacent the disc.

Other aspects and methods are described and claimed.

### BRIEF DESCRIPTION OF DRAWINGS

The invention will be described in detail below by referring to the accompanying drawings:

FIG. 1 illustrates a simplified view of two spinal vertebrae.

FIGS. 2a-2c illustrate the preferred embodiments in connection with the spinal items shown and described in connection with FIG. 1.

FIG. 2d illustrates the preferred embodiment of FIGS. 2a-2c but without the spinal items for sake of discussion.

FIG. 3 illustrates a generally cross-sectional view of an additional preferred embodiment, consistent with the preceding and including an anulus entry device.

### DESCRIPTION OF EMBODIMENTS

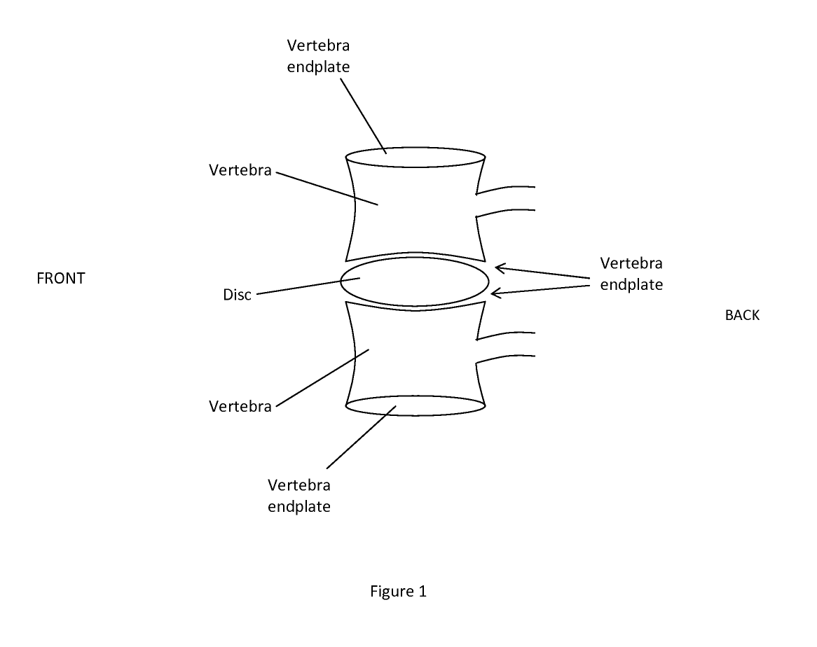

FIG. 1 was discussed in the Background Art section of this document and the reader is assumed to be familiar with the concepts of that Figure and discussion.

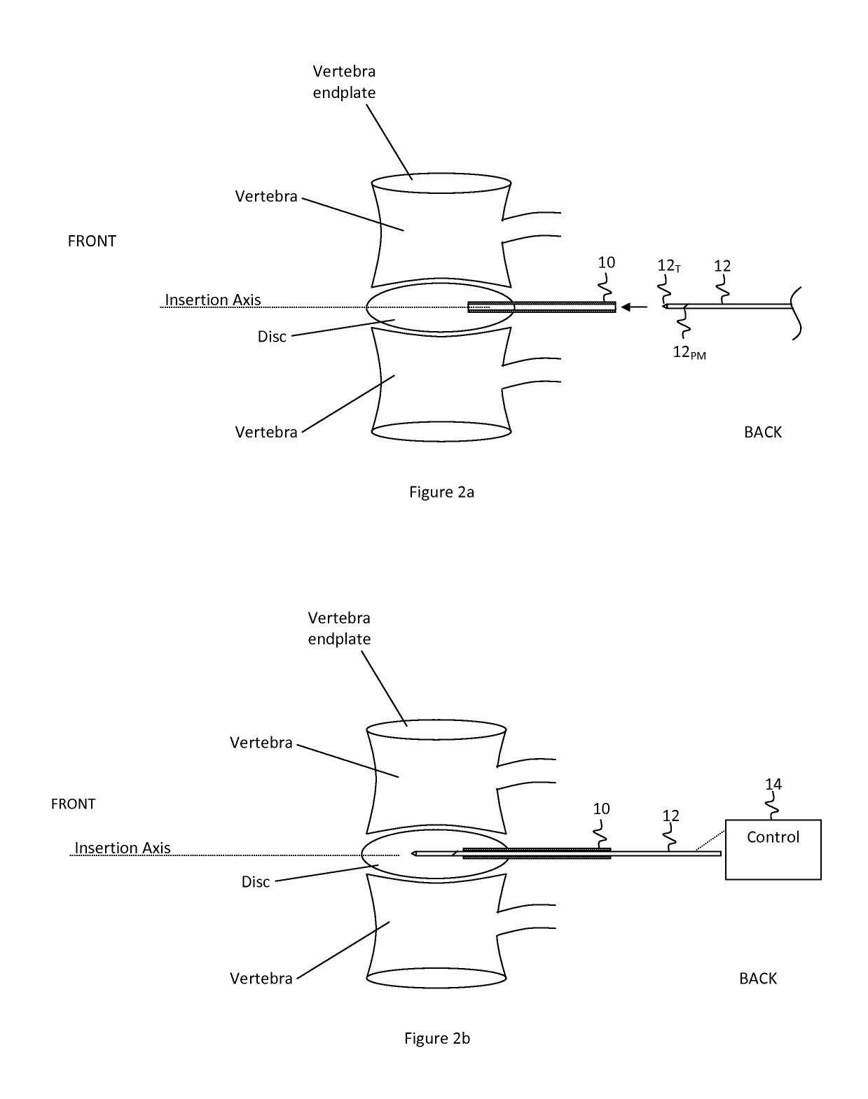

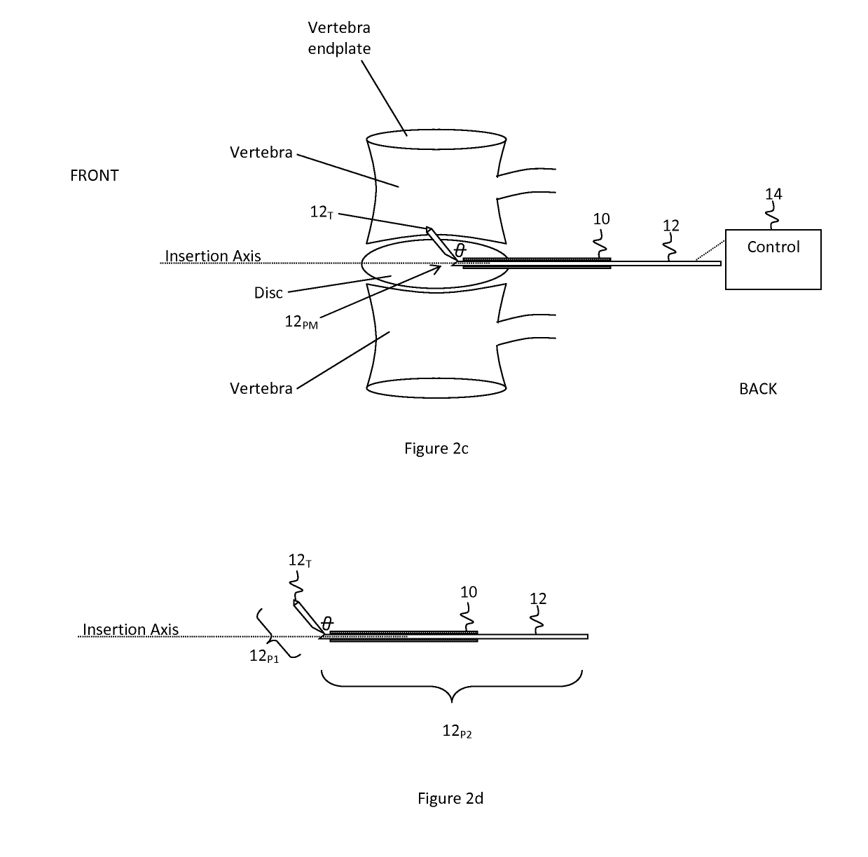

FIGS. 2a-2c illustrate preferred embodiments in connection with the spinal items shown and described in connection with FIG. 1. In FIG. 2a, a pathway is introduced into the disc by way of an apparatus 10, which is shown in cross-section and may be a cannula, as is known in the medical arts. Cannula placement is well known and may involve the use of related apparatus and steps, including percutaneous entry, a guide wire, and dilitation. In various preferred embodiments and based on various considerations including location of the spinal treatment and the underlying medical condition, preferred embodiment methods for locating the apparatus 10 for treatment according to this document may include posterior insertion, posterior lateral (e.g., 30 to 60 degrees off midline) insertion, direct lateral insertion, or anterior insertion.

The inner diameter of the cannula is preferably in a range of 1.0 to 3.0 millimeters, and it provides a pathway cavity through which apparatus and fluids may pass. In this regard and more pertinent to the preferred embodiments, the pathway created by the apparatus 10 permits placement along an insertion axis of an additional penetrating device 12, which in FIG. 2a is shown prior to such insertion and in FIG. 2b is shown inserted through the apparatus 10. In a preferred embodiment, the penetrating device 12 may be formed, or may include or have at surfaces, of various materials, including, as examples, medical grade steel, titanium, cobalt chromium, or nitinol. Additionally, in FIG. 2b, therefore, a leading tip 12_{T} (labeled in FIG. 2a, by example, to simplify other drawings), of the penetrating device 12, is placed through the inner pathway of the apparatus 10 so that ultimately the tip 12_{T} extends beyond that pathway and into the interior of the disc. Note also that for certain locations of the spine (e.g., sacral or lower lumbar), the apparatus 10 may have a shape that is curved, in whole or part.

A control unit 14 is also shown in FIGS. 2b and 2c, in connection with the penetrating device 12. The control unit 14 includes any manner of electrical and/or mechanical guidance and signal control so as to effect the steps described in this document. In this regard, the combination of the penetrating device 12 and the control unit 14 allow an additional key aspect, as illustrated in FIG. 2c. Specifically, in FIG. 2c, and once the penetrating device 12 has its tip 12_{T} located in the disc, then either through mechanical and/or electrical manipulation, the tip 12_{T} of the penetrating device 12 is operated to shift, either upward or downward, at an angle θ from the longitudinal axis of the remainder of the penetrating device 12 that is still positioned through the apparatus 10. Indeed, by way of example, note that the penetrating device 12 is shown in all three FIGS. 2a-2c to include a pivoting mechanism 12_{PM} near its tip 12_{T} (both labeled in FIG. 2c), so that once the device 12 is inserted as shown in FIGS. 2b and 2c, its tip 12_{T} may pivot about the mechanism 12_{PM} so as to achieve the angular displacement of θ as shown in FIG. 2c. By pivoting in this manner, the tip 12_{T} of the penetrating device 12 makes contact with the adjacent vertebral endplate, which in the example of FIG. 2c is the endplate above the disc. Note also while not shown, the pivoting mechanism 12_{PM}, as well as other aspects included with the penetrating device 12 and/or the control unit 14, are further operational for controlling the depth of penetration of the tip 12_{T} into both the disc and the vertebral endplate.

Various manners of implementing the pivoting mechanism 12_{PM} are contemplated within the inventive scope. For example, while not shown, a gearing mechanism may be included at the point, to include a worm gear and/or ratcheting teeth, so as to change the angular displacement of θ. Moreover, in one preferred embodiment the displacement of θ may be continuously variable, while in another it may have fixed selectable values or increments. Note also that the pivoting mechanism 12_{PM} is only by way of example, where other manners may be implemented so as to allow a tip or extension to reach in a direction away from either a lateral orientation or away from the insertion axis of the apparatus and toward a vertebral endplate.

Further in connection with FIG. 2c, note that the tip 12_{T}, extending away from the insertion axis, includes a sufficient apparatus so as to penetrate the endplate and thereby extend within the interior of the vertebra. This apparatus may include, for example, an awl or a drill, where the former by its shape and with applied force may be sufficient for penetration, and where the latter for example might be operated in connection with the control unit 14 to thereby accomplish stimulation to cause rotation of the drill, and hence the tip 12_{T}, so as to accomplish a rotational assist with such penetration. Other approaches also are contemplated within the inventive scope, including a punch or a cut. Moreover, differing preferred embodiments use a material and/or structure selected for the tip 12_{T} so as to facilitate its intended functionality of penetrating a vertebral endplate. For example, the tip 12_{T} may comprise a hardened material, a diamond coating, and/or may be constructed using atomic sharpening.

Given the preceding, the pivoting (or direction changing) functionality of the tip 12_{T} of the penetrating device 12, and the penetration of the vertebral endplate, allow one or more holes (i.e., apertures) to be formed into the disc endplate. These holes will facilitate treatments that may be akin to what is known as microfracture surgery, which is typically performed in connection with treating damaged cartilage in arthroscopic knee surgery. In such microfracture surgery, a tool is used to create small holes in bone adjacent the knee cartilage and to penetrate to a sufficient depth to reach a blood supply and cells (e.g. including stem cells). These biological materials may then get to the surface layer and stimulate nearby cartilage growth. Due to the maneuverability in the location and structure of the knee, a linear tool may be used to create such holes. In contrast, however, the preferred embodiments are directed to usages in connection with the spine, where physical manipulation of a linear tool may not be feasible, particularly in the lumbar region. Instead, the use of a preferred embodiment as depicted in FIGS. 2a-2c, where the tip 12_{T} may be directed in a direction that is not co-linear with the length of the tool that is inserted into the back region, thereby facilitates placement of the hole-causing tip into the vertebral endplate. With such holes, proper extraction of bone marrow elements, including stem cells, and usage of attendant biologics, may be implemented so as to have certain chemistries move into or be available in the areas of the intervertebral disc. As a result, it is projected that such chemistries may assist with healing and growth in the area so as to augment or supersede existing treatment modalities.

FIG. 2d again illustrates the penetrating device 12, but without the spinal elements so as to simplify the drawing and for purposes of some additional observations. Having described the preferred embodiment functionality of penetrating a vertebral endplate, more generally one skilled in the art should now recognize that the penetrating device includes two portions, a first portion 12_{P1} that is first inserted into the skin along the insertion axis, through the disc, and ultimately departs from the insertion axis to reach and penetrate the endplate. Following the first portion 12_{P1} is a second portion 12_{P2}, which provides additional lateral and angular reach of the first portion 12_{P1}. The second portion 12_{P2} also may provide electrical support to the first portion first portion 12_{P1}, for example, where the first portion first portion 12_{P1} implements a drill bit, or where the pivoting mechanism 12_{PM} requires an electrical stimulus so as change the angle as between the first portion 12_{P1} and the second portion 12_{P2}. Moreover, the second portion 12_{P2} also may provide mechanical force to the first portion 12_{P1}, for example, where the first portion implements an awl, punch, or cutting tip that requires force to be applied via the second portion 12_{P2}, so as to drive the tip 12_{T} of the awl into the vertebral endplate. Still further, both portions 12_{P1} and 12_{P2} may support further irrigation and/or extraction in the area of surgery and injury, consistent with the methodology described above. Given these observations, the preferred inventive scope may include various different configurations, such as the angular pivoting mechanism as described, an arcuate shape of part of the penetrating device 12, and possible a telescopic portion as well. Still other options are contemplated within the inventive scope.

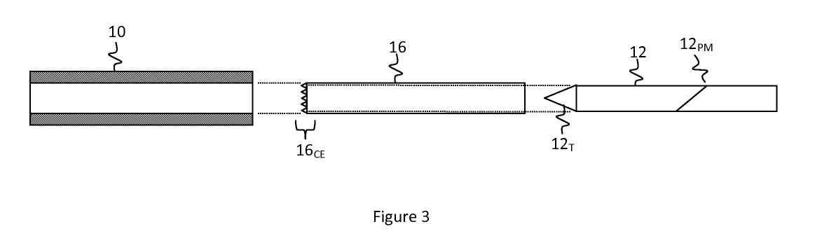

FIG. 3 illustrates a generally cross-sectional view of an additional preferred embodiment, consistent with the preceding and adding an anulus entry device 16, as now described. First, note that FIG. 3 again illustrates the apparatus 10 (e.g., cannula) and the penetrating device 12, as described above, and in enlarged form versus previous Figures so as to illustrate additional aspects. According to this preferred embodiment, however, the anulus entry device 16 is included so as also to fit within the apparatus 10, by having an outer diameter less than the inner diameter of the apparatus 10. Moreover, the anulus entry device 16 is included for purposes of first piercing or cutting through the outer portion of the spinal disc, prior to entry into the disc of the tip 12_{T} of the penetrating device 12. Specifically, it is recognized in connection with the preferred embodiments that a spinal disc includes an outer portion, known as an anulus (or annulus fibrosus), which surrounds the disc interior, namely, the nucleus (or nucleus pulposus), and where the former includes materials that are generally more fibrous than the latter. In use of the apparatus of FIG. 3, therefore, the apparatus 10 is located through the skin and its end adjacent the disc to be treated, and next the anulus entry device 16 is passed through the interior of the apparatus 10 so that a cutting edge 16_{CE}, of the anulus entry device 16, contacts the disc anulus. Next, the cutting edge, 16_{CE} is manipulated or operated so as to create an aperture in the disc anulus, such as by piercing, puncturing, or cutting and/or rotation, potentially in combination with control unit 14. After the aperture is so formed, the penetrating device tip 12_{T}, and a portion of the remainder of the penetrating device, is passed through that aperture. In one preferred embodiment, the penetrating device 12 has an outer diameter less than the inner diameter of the anulus entry device 16, and the anulus entry device 16 is hollow, so that the penetrating device 12 may pass inside of, and through, the anulus entry device 16, without removing the latter. In another preferred embodiment, after the anulus entry device 16 creates the disc aperture, the device 16 is removed from the apparatus 10, and then the penetrating device 12 is passed through the same apparatus 10, as described in connection with earlier Figures. As yet a third preferred embodiment, separate penetrating surfaces may be created on a single member 12 for passing through the apparatus 10, with a first surface for penetrating the disc anulus and a second surface for penetrating the vertebral endplate. Each implementation may have advantages, as will be appreciated by one skilled in the art. In any event, as also described above, once the penetrating device 12 is inside the disc, its tip 12_{T} is moved in a direction off the insertion axis (see FIGS. 2c and 2d), so as to penetrate a vertebral endplate, adjacent the penetrated disc.

Given the above, the inventive scope contemplates apparatus and methodology for forming an aperture in an intervertebral disc so as to permit bone marrow elements to move into the region of an adjacent intervertebral disc. These embodiments may provide numerous benefits over current manners of spinal disc treatment. For example, such embodiments may have a profound impact on reducing existing treatments while achieving comparable if not increased efficacies. Such benefits also may, for example, reduce the need for medication as well as the complexity and potential drawbacks of certain existing surgical approaches. Thus, numerous benefits have been suggested, and still others will be appreciated by one skilled in the art. Further, while the inventive scope has been demonstrated by certain preferred embodiments, one skilled in the art will appreciate that it is further subject to various modifications, substitutions, or alterations, without departing from that inventive scope. For example, while certain pivot approaches and shapes have been provided, alternatives may be selected. Thus, the inventive scope is demonstrated by the teachings herein and is further guided by the following exemplary but non-exhaustive claims.

What is claimed is:

1. A surgical apparatus for use in connection with spinal surgery, the surgical apparatus comprising:
a first portion having a tip for first following along an insertion axis, for entering into a spinal disc, and for penetrating an endplate of a vertebra adjacent the spinal disc; and
a second portion, adjacent the first portion, for remaining along the insertion axis as the tip of the first portion extends to penetrate the endplate of the vertebra adjacent the disc; and
apparatus for changing an angle between the first portion and the second portion.

2. The surgical apparatus of claim 1 wherein the first portion tip comprises an awl tip.

3. The surgical apparatus of claim 1 wherein the first portion tip comprises a drill tip.

4. The surgical apparatus of claim 3 wherein the second portion comprises electromechanical support of the drill tip.

5. The surgical apparatus of claim 1 wherein the first portion tip comprises a punch tip.

6. The surgical apparatus of claim 1 wherein the first portion tip comprises a cutting tip.

7. (canceled)

8. The surgical apparatus of claim 1 wherein the apparatus for changing an angle is for changing a continuously variable angle.

9. The surgical apparatus of claim 1 wherein the apparatus for changing an angle is for changing between preset selectable angles.

10. The surgical apparatus of claim 1 wherein the tip of the first portion extends to penetrate an endplate of the vertebra adjacent the disc.

11. The surgical apparatus of claim 1 wherein the first portion and the second portion form a first member, and further comprising a second member for forming an aperture in the spinal disc, wherein the first portion is for entering into the aperture.

12. The surgical apparatus of claim 1 wherein the first member passes through an interior of the second member.

13. The surgical apparatus of claim 1 wherein at least one of the first portion and the second portion further comprises means for irrigation.

14. The surgical apparatus of claim 1 wherein the first portion comprises:
a first surface for entering into a spinal disc; and
a second surface for penetrating an endplate of a vertebra.

15. A method of spinal surgery, the method comprising:
inserting an apparatus into a body and directed the apparatus along an insertion axis toward a spinal disc, the apparatus comprising a first portion and a second portion;
directing the first portion, having a tip, for entering into a spinal disc and for penetrating an endplate of a vertebra adjacent the spinal disc; and
during the directing step, retaining the second portion, adjacent the first portion, along the insertion axis as the tip of the first portion extends to penetrate the endplate of the vertebra adjacent the spinal disc.

16. The method of claim 15 and further comprising, after the directing step and prior to penetrating the endplate of the vertebra, changing an angle between the first portion and the second portion.

17. The method of claim 15 wherein the step of inserting an apparatus into a body comprises inserting the apparatus through a cannula.

18. The method of claim 17 and further comprising, prior to the directing step, directing an anulus apparatus through the cannula and for forming an aperture in an anulus of the spinal disc.

19. The method of claim 18 wherein the step of directing the first portion comprises directing the first portion through an interior of the anulus apparatus.

20. The method of claim 15 wherein the inserting step comprises posterior insertion.

21. The method of claim 15 wherein the inserting step comprises posterior lateral insertion.

22. The method of claim 15 wherein the inserting step comprises direct lateral insertion.

23. The method of claim 15 wherein the inserting step comprises anterior insertion.
